(19) United States
(12) Patent Application Publication
(10) Pub. No.: US 2026/0041629 A1
(43) Pub. Date: Feb. 12, 2026

(54) Methods and Systems to Reduce Injury in Treated Tissues

(71) Applicant: MileHigh Repro Solutions, LLC, Fort Collins, CO (US)

(72) Inventors: Myles J. Shepherd, Carr, CO (US); Lisa A. Herickhoff, Fort Collins, CO (US); Joaquin Crego, Wellington, CO (US); Justin Crego, Wellington, CO (US); Larry Crego, Fort Collins, CO (US); Hannah Mabry, Fort Collins, CO (US); Jim A. Herickhoff, Fort Collins, CO (US)

(21) Appl. No.: 18/847,140

(22) PCT Filed: Mar. 14, 2023
(86) PCT No.: PCT/US2023/015203

§ 371 Date: Sep. 13, 2024

Related U.S. Application Data
(60) Provisional application No. 63/319,866, filed on Mar. 15, 2022.

(51) Int. Cl. A61K 9/00; A61K 9/70; A61K 31/192; A61K 31/201; A61K 31/353; A61K 31/395; A61K 31/60; A61P 1/06; A61P 21/02

(52) U.S. Cl. CPC A61K 9/0034; A61K 9/7015; A61K 31/192; A61K 31/201; A61K 31/353; A61K 31/5545; A61K 31/60; A61P 1/06; A61P 21/02

(57) ABSTRACT

Embodiments of the application provide methods and systems for the relaxation of target tissues (10) of mammals (1) using a treatment (2) having a natural substance (3). A natural substance may be a plant-based or even an algae-based substance. A treatment may be applied to a target tissue with a treatment applicator (8) and even a delivery mechanism (11). A mammal may be positioned perhaps with an elevation mechanism (13) for exposure of the target tissue to a treatment.

### CROSS-REFERENCE TO RELATED APPLICATIONS

This is a PCT International Patent Application claiming the benefit of and priority to U.S. Provisional Application No. 63/319,866 filed on Mar. 15, 2022, hereby incorporated by reference herein in its entirety.

### STATEMENT REGARDING FEDERALLY SPONSORED RESEARCH OR DEVELOPMENT

Some of the work in this application relates to work performed under Grant from USDA NSIIC awarded in December 2021. The US government may have certain rights in this inventive technology, including “march-in” rights, as provided for by the terms of 35U.S.C. § 203.

### TECHNICAL FIELD

The application generally relates to systems and methods for aiding passage of a device through a restricted orifice without substantial physical damage. As such, embodiments may relate to systems and methods for preparing a reproductive organ, tissues, or even cells, for breeding or related technologies, or for other methodologies requiring passage through a restricted orifice. The systems and methods disclosed herein can enable the use of artificial reproductive technologies and even to improve outcomes of these technologies via increased ease of procedure.

### BACKGROUND

Reproductive organs, like other bodily organs, may be subjected to biological, mechanical and physiological restrictions that may decrease the ability to function in a traditional breeding situation or even in an assisted reproductive breeding situation. Therefore, preparing a reproductive tract may be important to achieve breeding success whether using natural or artificial means, but perhaps may be even more important with artificial means. Assisted reproductive technologies may include collection of reproductive cells, in vitro handling of cells, transfer of cells or embryos, retrieval of oocytes, cryopreservation or vitrification of cells or tissues, artificial insemination intravaginally, intracervically, or perhaps even laparoscopically, retrieval of embryos at various developmental stages, transfer of cells or tissues into a recipient, and the like, perhaps using commonly understood technologies.

Such systems and methods for preparation may be inclusive of both the female and male reproductive tracts. These preparations may include the readying of one, or even a plurality of portions of the female tract, including the vagina, cervix, and uterus as well as the readying of the male tract: prepuce, glans, penis, testes, and epididymis. Readying may include actual preparation for said treatment, but also preparation to avoid injury, infection, inflammation or similar damage. Unfortunately, the anatomy of some reproductive tracts may require that more invasive and even damaging techniques be utilized in such preparation, which may be accompanied by the increased potential for damage and infection.

It is important to manage and maintain the health of the mammal's reproductive organs. Indeed, damage to one or multiple aspects of a male or female tract can result in low or no fertilization or conception of that particular insemination event, or may result in abortion of a pregnancy. At worst, it may result in partial or even complete infertility. It is important to consider that both the male and female aspects are important for successful breeding but, for a non-limiting example, if sperm are delivered in a way that damages the female reproductive organs, the results can be poor at a minimum, or mortal at worst. Poor fertility from insemination events may cause not only physiologic distress, emotional distress, but may cause financial distress. This is true whether in livestock, where reproduction is the foundation of herd propagation or perhaps in humans experiencing infertility treatments.

As but one non-limiting example, current female technologies may rely on harsh procedures such as laparoscopic artificial insemination or similar invasive procedures relating to the delivery of cells to the appropriate area of the reproductive tract. Currently, laparoscopic insemination may be the only repeatable and reliable option for small ruminant breeding such as the ewe (for example, female sheep), doe (for example, female goat or deer), or cow (female elk or bovine) or other wild ungulates. The reliance on laparoscopic insemination may be caused by, or even exacerbated by, the complex physiology and anatomy of the reproductive tract and even the cervix of small ruminants. This can be further exacerbated by the physical inability to grasp the tissues through the anus as one does with bovine. The risk of laparoscopic AI may include infection, damage to the reproductive tract, delivery of semen to the wrong area perhaps rendering the technique ineffective, low pregnancy rates, inflammation, and the like. Finally, the apparent sensitivity of some animals including small ruminants, to anesthesia, and the trauma imparted by the laparoscopic insemination procedure may cause pain and/or inflammation and can increase the odds of a negative outcome including death. Laparoscopic techniques may be required whether one is inseminating, obtaining oocytes, or transferring embryos in the above example.

Methods which may reduce the need for laparoscopic AI include transcervical insemination or natural breeding. Unfortunately, the transcervical method of insemination or embryo transfer is not commonly used in small ruminants and other species because of the poor breeding outcomes, including very low conception rates. Hyaluronic acid has been tested to achieve passage of tissues without damage but the effects were systemic, slow to react, and long lasting perhaps then making it largely impractical in any commercial sense. Similarly estrogen and al-adrenergic antagonists such as prazosin and tamsulosin (e.g., FloMax® tamsulosin HCL medication) have been assessed and found to require too long of a treatment period to be effective, or may have extended treatment effects. Physical items such as hemostats have been tested but may be excessively painful, may have caused animal distress, or may even cause long term damage. In another attempt, analgesics or anesthetics such as lidocaine, have been tested but do not relax tissue and only blocks pain signals.

Natural breeding can significantly limit rapid genetic gain required for livestock. Moreover, it does not enable out-of-season breeding, and can possess potential biosecurity concerns such as sexually transmitted diseases. Current attempts at cervical insemination can lead to permanent or temporary tissue damage, inflammation, swelling and perhaps even infertility.

A technology that can relax tissue without adverse effects has many different potential applications. In addition to breeding efforts, reproductive tissues often must be disturbed for purposes such as regular health examination, placement of intrauterine devices, surgical intervention, cervical softening and/or perhaps ripening prior to parturition. This is true for other, similarly constructed tissues with smooth muscle and high collagen content, such as sphincters and ductal tissues. Any method which could reduce damage and ease manipulation would be valuable in clinical settings.

### DISCLOSURE OF INVENTION

The present application includes a variety of aspects, which may be selected in different combinations based upon the particular application or needs to be addressed. In various embodiments, the application may include a solution that can enable cervical breeding in difficult to breed mammals, decrease infection, decrease bodily trauma, and perhaps even improve pregnancy results.

It is an object of the application to provide maximal reproductive success perhaps with a non-sedative, minimally invasive, reproductive-cell-compatible, and even low-antibiotic-use insemination methods and systems.

It is another object of the application to provide methods and systems for improving cervical insemination outcomes by preparing the female reproductive tract. This may include a composition for application, a method for applying the composition, and even a delivery mechanism for the system. Such may minimize the chance of injury perhaps by enabling maximum tissue manipulation, optimizing physiological and anatomical factors, and may decrease physical discomfort and trauma.

It is yet another object of the application to improve transcervical insemination repeatability and reliability while mitigating risks of pain, infections, high morbidity, and even mortality risk. This may provide an increase in breeding success and a sustainable future for certain species to which the technologies may be applied. This may include a composition that can result in a maximum ability to transverse, navigate or otherwise manipulate tissues using common insemination tools. Such composition may initiate relaxation of tissue, may induce relaxation, ripening, softening, or cause other related physical impacts of the cervix. Such impacts may mimic some physiological responses such as estrus or may enhance the estrus response.

It is another object of the application to provide options for relaxation of tissues for the cervix and other tissues such as those having a limited or even small opening that may dilate, or perhaps relax, become less restrictive, or otherwise enlarge, and through which one may desire passage of an instrument, probe or similar device, or perhaps even a fetus.

It is an object of the application to provide applications for tissue relaxation in insemination, parturition, physical examination, passage of instrumentation, removal of foreign materials or devices, retrieval of reproductive cells, addition of reproductive cells, addition of embryos, tissues or perhaps addition of foreign materials or devices, and the like.

It is yet another object of the application to identify a specific location or a general location near a desired opening and perhaps within an area of desired passage for treatment. Embodiments may relate to the application of a solution as well as the delivery mechanism for the application device. Such a delivery mechanism may be used to insert or even apply a solution or a solution containing applicator. This application may be applied to a specific location or even at a general location perhaps near a cervical opening within a vagina, vaginal vault, vestibule, or the like.

It is an object of the application to provide a solution, compound, composition, formulation or the like perhaps for cervical ripening, relaxation, and or softening through the specific location.

It is another object of the application to provide a material, device, or perhaps an absorbent material that may contact al tissue and can enable transfer of a composition from the delivery device to the tissue or surface.

It is yet another object of the application to provide a device, procedure, injectable mechanism, placement mechanism, or the like that can enable a correct placement of a device. A device may be of a size and shape as to properly fit within a desired locational of impact. A desired location may include a vagina, vestibule, cervical opening, or the like. Regardless of area of impact, the placement mechanism may provide proper and even sufficient epithelial contact for the desired effects.

It is an object of the application to provide a mechanism for affecting an appropriate response perhaps including physical positioning, a timing mechanism, appropriate spatial location device, or the like.

It is another object of the application to provide a methodology to enable repeatability of a process with high accuracy, precision, and even minimal manipulation.

It is yet another object of the application to provide a positioning device that can enable or even ease an ability to deliver a device, solution, or even related materials. A positioning device may be applied to the entire physical body of the treated being, may affect a change only in the organ or tissue to be treated, or perhaps even may affect proper positioning of both the body and the tissue, organ, or the like. A positioning device may be utilized to ensure the reliability of the delivery mechanism, of the solution, or even of the combined system.

In embodiments, systems and methods may include a solution, an applicator for the solution, a delivery instrument for an applicator, a removal method for an applicator, a positioning device, use of other methodologies, and the like.

Naturally, further objects, goals and embodiments of the application are disclosed throughout other areas of the specification, claims, and drawings.

### BRIEF DESCRIPTION OF THE DRAWINGS

FIG. 1 is a non-limiting example of a delivery mechanism device with a treatment in accordance with some embodiments.

FIG. 2 is a non-limiting example of application of a treatment with a target tissue in accordance with some embodiments.

FIG. 3A is a non-limiting example of positioning mammals and an elevation mechanism utilizing a variable-height platform in accordance with some embodiments.

FIG. 3B is a non-limiting example of positioning mammals and an elevation mechanism utilizing a variable grade in accordance with some embodiments.

FIG. 3C is a non-limiting example of positioning mammals and an elevation mechanism utilizing a tilt-table-like device in accordance with some embodiments.

FIG. 3D is a non-limiting example of positioning mammals and an elevation mechanism utilizing an abdominal sling and foot support in accordance with some embodiments.

FIG. 4 shows experimental data of AI Depth by Treatment in accordance with some embodiments.

FIG. 5 is a non-limiting example of a work flow in accordance with some embodiments.

FIG. 6A shows a graph of the percentage of cervical rings passed in different treatments in accordance with some embodiments.

FIG. 6B shows a graph of the ½cc AI rod depth of passage in different treatments in accordance with some embodiments.

FIG. 7 shows a graph of AI rod cervical penetration time series in accordance with some embodiments.

FIG. 8 shows a non-limiting example of a hip elevation angle in accordance with some embodiments.

FIG. 9 shows a schematic example of a treatment in a physical state in accordance with some embodiments.

### MODE(S) FOR CARRYING OUT THE INVENTION

It should be understood that embodiments include a variety of aspects, which may be combined in different ways. The following descriptions are provided to list elements and describe some of the embodiments of the application. These elements are listed with initial embodiments; however, it should be understood that they may be combined in any manner and in any number to create additional embodiments. The variously described examples and preferred embodiments should not be construed to limit the embodiments of the application to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the application may include methods for preparing tissues comprising the steps of providing a target tissue (10) of a mammal (1); topically applying a treatment (2) comprising a natural substance (3) to said target tissue; and perhaps even temporarily relaxing said target tissue with said treatment. It may include methods for treating mammals comprising the steps of providing a mammal; providing a target tissue of said mammal; elevating hips (16) of said mammal above its shoulders (17) to position said target tissue of said mammal, wherein said hips are elevated at an angle between about 5 to about 50 degrees above said shoulders; and perhaps even applying a treatment to said target tissue.

Methods may include treating tissues comprising the steps of providing a target tissue of a mammal; providing a treatment applicator containing a treatment having a natural substance; positioning said mammal to enable said treatment; topically applying said treatment having said natural substance to said target tissue from contact of said applicator with said target tissue; removing said applicator from said target tissue of said mammal; and perhaps even temporarily relaxing said target tissue with said treatment. Other methods may include relaxing tissues comprising the steps of providing a target tissue of a mammal, said target tissue chosen from cervix, anus, vagina, and uterus; providing a treatment applicator containing a treatment having a natural substance; holding said treatment applicator containing said treatment with a delivery mechanism; placing said treatment applicator containing said treatment on or near said target tissue with said delivery mechanism; topically applying said treatment to said target tissue from contact of said treatment applicator with said target tissue; removing said treatment applicator from said target tissue of said mammal with said delivery mechanism; and perhaps even temporarily relaxing said target tissue with said treatment.

Systems may include a treatment configured to temporarily enable passage through a target tissue of a mammal, said treatment comprising a natural substance (3). A mammal treatment system may include an elevation mechanism (13) configured to elevate hips of a mammal above its shoulders to expose a target tissue for treatment of mammal; wherein said elevation mechanism is configured to elevate said hips at an angle between about 5 to about 50 degrees above said shoulders.

A tissue treatment system may include a treatment applicator (8) containing a treatment having a natural substance (3); wherein said treatment is configured to temporarily relax a target tissue of a mammal. Systems may include a tissue treatment system comprising a treatment applicator containing a treatment having a natural substance; a delivery mechanism configured to place said treatment applicator on or near a target tissue of a mammal; wherein said treatment is configured to temporarily relax said target tissue.

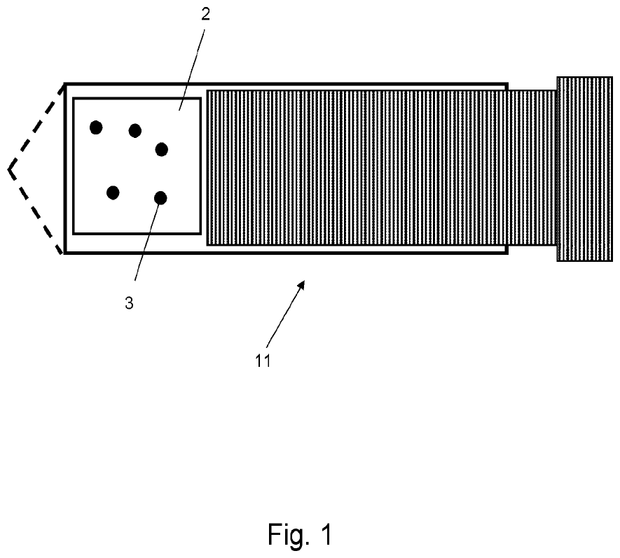

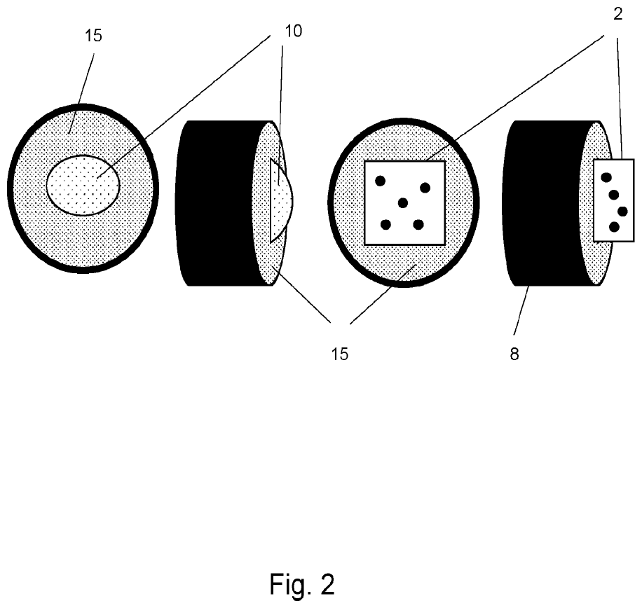

Embodiments of the application may provide methods and systems for treatment of tissues perhaps to reduce injury and even allow ease in a procedure. It may include abatement of injury during manipulation, treatment, modification, delivery, breeding events, or the like with mammals. As may be understood from FIGS. 1 and 2, a treatment (2) may be applied perhaps even topically applied to a target tissue (10) of a mammal. A treatment may include plant-based or even algae-based substances (3). A target tissue (10) and even a surrounding tissue (15) may come into surface contact with a treatment (2) perhaps via a treatment applicator (8).

A treatment (2) may be a composition of materials, solutions, extracts, chemicals, compounds, or the like that may affect a target tissue (10) perhaps so that a manipulation event to the target tissue can occur without bruising, tearing, any other harm, or the like to the target tissue surrounding the area of manipulation. A treatment may temporarily relax a target tissue after application thereof. This may provide softening, loosening up, make less tense, opening, or the like of a tissue after application of a treatment. In some embodiments, an affect from a treatment on a target tissue may include but is not limited to: lubrication, hydration, cause mucosal production, a positively affected redox balance, dilation, opening, relaxation, relaxation of muscles or tissues, ripening of a tissue, any combination thereof, or the like. Temporarily relaxing of a target tissue may allow expansion of a target tissue and even allow passage through a restricted orifice of a mammal. As but one non-limiting example, a treatment may be used to relax reproductive tissue perhaps enabling manipulation of tissues during insemination events without damage. In embodiments, a physiological response of a mammal to a treatment may not be dependent on hormonal influences of the mammal.

A treatment may be made of or include a natural substance (3). A natural substance (3) may be a plant-based or even algae-based substance which may include, but it not limited to extracts derived from families or orders of plants or algae such as Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, any combination or permutation thereof, and the like.

A treatment may include other substances such as but not limited to: glycosylated phenolic compounds, anthocyanin, carotenoids, flavonoids, apocarotenoids, quercitin, isohamnetin (1) glycoside, triglycerides, tocopherols, salicylate, linoleic and linolenic acids, phenols, antimicrobial or antifungal agents, anti-inflammatory agents, antioxidants, any combination or permutation thereof, and the like.

In some embodiments, a treatment may include chosen from: antimicrobial agents, antifungal agents, anti-inflammatory agents, antiprotozoal agents, bacteriostatic agents, metal oxides, trace minerals, bromelain, papain, ricinoleic acid, thymol, carvacrols, antioxidants, phenolic acids, flavonoids, methylglyoxal peptides, carotenoids, carophyllene, beta carotene, apocarotenoids, vitamin C, vitamin E, linoleic acid, linolenic acid, terpene alcohols,, linalyl acetate, herniarin, coumarin, anthocyanidins, sesquiterpenes, terpenoids, tocotrienols, oxidative scavengers, cellulose, microcrystalline cellulose, methylcellulose, ethylcellulose, hydroxyethyl cellulose, hydroxypropyl cellulose, hydroxypropyl methyl cellulose, sodium carboxymethyl cellulose, semisynthetic cellulose, polyurethane foam, polyvinyl propylene, pectin, alginates, sodium alginates, polyvinyl alcohol (PVA) materials, soda lime, allochroic silica gel, activated carbon, anhydrous calcium chloride, iota-carrageenans, kappa-carrageenan, lambda-carrageenans, chitosan, hyaluronic acid, gelatin, gellan gum, xanthan gum, poloxamers, polyacrylates, Carbopol® polymer, polyvinylpyrrolidone, polyethylene glycol, sodium polyacrylate, and any combination or permutation thereof.

In some embodiments, a treatment may have a primary component are chosen from: glycosylated phenolic compounds, anthocyanin, carotenoinds, flavonoids, apocarotenoids, hyoscine butylbromide, aapocarotenoids cholecalciferol, linoleic acid, terpene alcohols, delta-9-tetrahydrocanabinol, cannabidiol, terpenoids, oleic acid, salicylic acid, gallic acid, ferulic acid, vanillic acid, magnesium, ricinoleic acid, linalyl acetate, carophyllene, eucalyptol, lavandulyl acetate, monoterpenoid phenols, p-cymene, γ-terpinene, protease enzymes, and any combination or permutation thereof. A treatment may have a secondary component chosen from: quercitin, isoha, mnetin (I) glycoside, triglycerides, tocopherols, medium chain triglycerides, oleic acid, palmitic acid, stearic acid, β-sitosterol, methyl salicylate, carophyllenes, phenolic acids, flavonoids, sterols, trace minerals, stearic acid, linolenic acid, limonene, camphor, lavandulol, terpenoids, camphene, pinene, papain-like proteases, and any combination or permutation thereof.

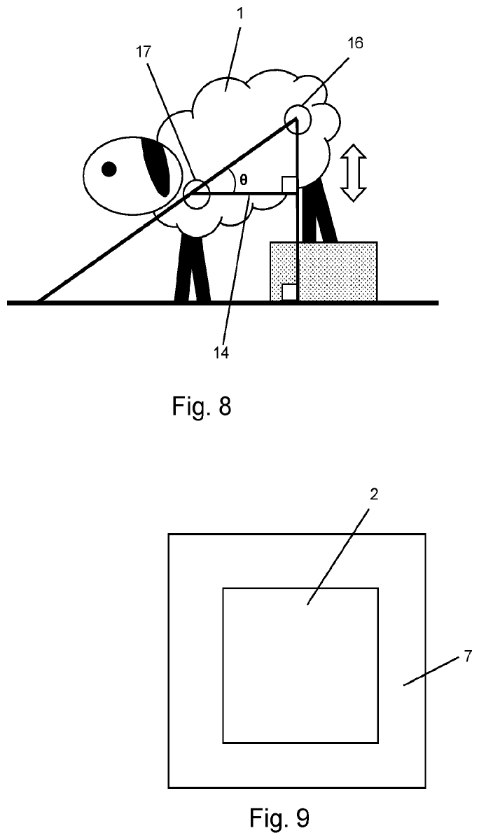

As may be understood in FIG. 9, a treatment may be used in any kind of physical state (7) including, but not limited to, liquid, semi-solid, solid, gel, cream, capsule, suppository, poultice, bioadhesive tablet, patch, film, ring, foam, polymer, mucoadhesive gel, any combination or permutation thereof, and the like. A treatment may be contained in a capsule, which may be dissolving, slow, or even timed-release, and a capsule may be made of a material such as wax, sugar, gel, glycerin, cellulose, collagen, any combination or permutation thereof, and the like.

A treatment may contain thickeners, stabilizers, or the like including but not limited to: pectin, alginates, guluronic acid, starch, carrageenans, any combination or permutation thereof, or the like. In some embodiments, a treatment may include a binder, a hydrophilic matrix-forming agent, or may be delivered as an in-situ gelling system formulation, or the like.

In embodiments, a treatment may be utilized in such a manner to maintain homeostasis perhaps without creating significant change in the environment of use. As a non-limiting example, a native pH of the environment, osmotic pressure, and perhaps even tissue hydration may be maintained and may not be substantially affected by a treatment. In some embodiments, multiple formulations of a treatment may be provided perhaps so that are slightly modified from one another and may be specified, modified, or even differently embodied to the particular tissue or location of use.

A target tissue (10) may be any kind of aggregate of similar cells and cell products including but not limited to constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, arterial tissue, and the like. A mammal (1) may be any kind of mammal including but not limited to an ungulate, a ruminant animal, a small ruminant animal, ewe, doe, cow, human, or the like or either domestic or wild origins.

Embodiments may include a treatment applicator (8). A treatment applicator may provide the capability to apply a treatment to a target tissue area. A treatment applicator (8) may include but is not limited to cotton, polyester, a natural material, a synthetic material, absorbent swab, cushion, sponge, sea sponge, pad, diaphragm, rubber convex device, pessary, capsule, any combination or permutation thereof, or the like. A treatment may be embedded or even contained in a treatment applicator.

In some embodiments, the material used to form a treatment applicator for a treatment may also contain or even be composed of one or more of the components of the treatment or other active ingredients or the like. A treatment applicator may be an absorbent swab, cushion, capsule, dissolving capsule, sponge, pad, diaphragm, rubber convex device, suppository, pessary, pellet, any combination or permutation thereof, or the like. A treatment applicator may be composed of one or a variety of materials including, but not limited to: seaweed, cotton, polyester, plastics, plastic-like, natural sponge, sea sponge, synthetic sponge, cellulose, microcrystalline cellulose, methylcellulose, ethylcellulose, hydroxyethyl cellulose, hydroxypropyl cellulose, hydroxypropyl methyl cellulose, sodium carboxymethyl cellulose, semisynthetic cellulose, polyurethane foam, polyvinyl propylene, pectin, alginates, sodium alginates, AbZorb fabrics, polyvinyl alcohol (PVA) materials, soda lime, allochroic silica gel, activated carbon, anhydrous calcium chloride, iota-carrageenans, kappa-carrageenan, and lambda-carrageenans, chitosan, hyaluronic acid, gelatin, gellan gum, xanthan gum, poloxamers, polyacrylates, Carbopol® polymer, polyvinylpyrrolidone, polyethylene glycol, sodium polyacrylate, other materials such as may be commonly known to those in the art, any combination or permutation thereof, or the like. Embodiments may be mixed, form an admixture, mixture, compound, or otherwise be combined with a treatment, may form a delivery mechanism, or the like.

A treatment applicator may include organic, natural, or even pharmacological products which can hold, absorb, and perhaps even apply a solution in an efficacious manner.

All components of a treatment, an applicator, or any of the materials perhaps used to enable effectiveness of application of a treatment may be sperm-compatible, embryo compatible, tissue compatible, organ compatible, or the like and may not cause irritation or even sensitization to the tissue within a location of use.

In embodiments, a treatment applicator and perhaps a form of a treatment may avoid systemic exposure. In another embodiments, a treatment applicator may not inhibit or even interfere with the functionality of the device for which the tissue is being treated. As a non-limiting example, a target tissue may be a cervix and a procedure to be conducted on the mammal may be the passing of an artificial insemination rod. A treatment may aid the efficiency and effectiveness of the procedure.

Embodiments of the application may provide a treatment and even a treatment applicator that can provide a desired residence time to a target tissue perhaps to enable functionality. A treatment applicator may enable an appropriate time of contact such as through erosion-based properties, removal, decomposition, a fast-dissolving insert, similar technology as may be known in the art, or the like.

In some embodiments, application of a treatment may be such that the treatment to be administered may be in one form but transformed to a second form upon contact with a target tissue and its conditions As but one non-limiting example, a treatment may be encased in a dissolvable capsule that may melt as it contacts the target tissue. Regardless of the format, and in some embodiments, the methodology and application, the case of use, dosing frequency, appropriate contact time, and functionality may be determined.

In some embodiments, a treatment applicator may embody a form that can be appropriate to the form of the treatment, the application, the specific tissue, the area of use, and perhaps even the desired response.

As may be understood in FIG. 1, a delivery mechanism (11) may be utilized which may contain a treatment (2) and perhaps even a treatment applicator (8). As a non-limiting example, a delivery mechanism may have a plunger or extension device and an outer casing which can be used to deliver a treatment applicator (8) containing a treatment (2) to a target tissue.

A treatment, a treatment applicator and perhaps even a delivery mechanism may be utilized in such a way as to avoid introduction of exogenous pathogens to the target tissue.

Application of a treatment may be used at an optimal location. A treatment which may be in a treatment applicator may be placed on or near a target tissue with a delivery mechanism (11).

A delivery mechanism may be an exterior device that can aid in the proper placement of a treatment or even a treatment applicator to a target tissue. A treatment applicator and a delivery mechanism may be of similar materials or may even be independent of one another. Alternatively, they may be of disparate materials.

In embodiments, a delivery mechanism may be configured to apply a treatment or a treatment applicator to a target tissue and may include but is not limited to a spray bottle, aerosolizer, mist device, syringe, tweezers, forceps, bimanual insertion, tenaculum forceps, pincher device, plunger device, clasping device, speculum, scoop, swab, an adjustable device, any combination or permutation thereof, and the like. A delivery mechanism may be adjustable, may be tapered such that one face may be a different size than another, may have one face that can be a different shape than another, or the like. Shapes may be flat, round, pointed, triangular, trapezoidal, rectangular, polygonal, bulbed, cupped, convex, concave, or the like.

A delivery mechanism may include a release and even an unseating aspect. A delivery mechanism may be capable of depositing a treatment or treatment applicator on or near a target tissue. A delivery mechanism may then also be capable of removing a treatment or treatment applicator from the target tissue. In embodiments, an unseating aspect may be a grasping device, an integral piece of the applicator, or perhaps even a retrieval device. This may include but is not limited to tweezers, forceps, pincher device, clasping device, a string, a suctioning device, a scoop, a swab, or the like. A delivery removal mechanism may include a manual step or even utilize internal bodily forces. It may be understood that a delivery removal or releasing device may be made of a disparate material than the delivery device. It may be included as part of the delivery device or may be a separate component.

In embodiments, a delivery mechanism may be made of any material or combination of materials, natural or not, and may even be of various lengths and even diameters such as are applicable to the particular space of a target tissue or treatment applicator. Use of a treatment applicator and/or a delivery mechanism may be such that it may not be inadvertently removed by other animals, inadvertently expelled before treatment is completed, or perhaps inadvertently removed by fence, or by other hazards that may be encountered by a treated mammal.

In embodiments, a delivery mechanism may consist of plastic, paper, metal, rubber, digital (fingers), other materials which may push, pull, squeeze, spread, drag, deliver, twist, crank or otherwise manipulate the device. A delivery mechanism may or may not be for individual use, reusable, or the like.

Embodiments of the system can be easy to use, relatively inexpensive, provide use in non-sterile environs, provide proper residence time to be efficacious for a tissue and its application, and perhaps can include instructions on improper usage and delivery.

In some embodiments, a treatment applicator and even a delivery mechanism may be sterilized, cleaned, otherwise disinfected, or the like and can then be reused.

A mammal having a temporarily relaxed target tissue may be treated with a procedure (36) such as but not limited to artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, laparoscopic surgery, or the like. Further procedures may include dilation, dilation of a sphincter muscle, an esophageal type application, an area of occlusion, an area of occlusion due to tissue over-growth, scar tissue, applications where it may be efficacious to gain passage into an area without causing damage, scaring, cutting, dislodging, or the like. Embodiments may be used in an examination of perhaps a prostate, passage of renal calculi, passing a salivary stone, ease of passing by a pyloric sphincter, and the like. It may be used to enable parturition when biological functionality can be failing. Other possible uses may include the examination or manipulation of any other soft tissues, in applications such as prostate examinations, uterine or cervical examinations or treatment, assisting in passing renal calculi through a sigmoid flexure or sphincter, easing passage of salivary stones, endoscopy, or other similar procedures. It may be used to relax ducts, such as perhaps tear ducts, may be used in the insertion of breathing or feeding tubes, in sinus inspections, to aid in relaxation of scar tissue, affecting change in smooth muscle, perhaps even aiding in relaxation of venous or arterial tissues.

Temporarily relaxing a target tissue may provide the relaxation of the target tissue for an amount of time which may include but is not limited to up to: about 20 minutes, up to about 30 minutes, up to about 60 minutes, up to about 180 minutes, or the like. A treatment may be applied to a target tissue for an amount of time which may include but is not limited to: about 15 minutes, about 30 minutes, about 60 minutes, about 180 minutes, or the like.

Embodiments may be for use in mammals (1), which may include but is not limited to four-legged mammals, sheep, goats, deer, elk, humans, and similar mammalian species perhaps where there may be a need for cervical or other soft tissue manipulation without trauma such as artificial insemination without damage to the cervix. Similarly, it may be utilized in any artificial breeding event. It may be used in parturition or birthing events, surgery situations, during placement of a device or compound, may provide access for examination or treatment, or in similar situations. Use of the various embodiments herein may preclude the need for use of an endoscope, other visualization equipment, or the like.

Embodiments of the application may require mechanical or even digital (electronic) control for a delivery mechanism. Embodiments may include a sensor for pressure measurement and even a pressure sensitive device.

Embodiments of the application may provide positioning of a mammal for application of a treatment or even for procedures conducted after relaxation of tissues. Utilizing treatments, treatment applicators, and perhaps even delivery mechanisms may include optimal positioning of a mammal for administration of a treatment and perhaps even a technique for positioning the mammals. Positioning may use a mammal positioner (12) which may be a restraining mechanism perhaps to temporarily immobilize the mammal during application of the tissue treatment. A patient to which a treatment applicator may be applied could be positioned such that the target tissue may be most easily contacted. In embodiments, a mammal positioner (12) may help to place a mammal into a desired position. A mammal positioner may incorporate restraining components such as harnesses, slings, bars, rails, chest or limb straps, headcatch such as may be found commonly in livestock handling chutes, or the like.

An example of a positioner may be an elevation mechanism (13) which may elevate or may even position limbs and a mammal's body such that an ideal arrangement may be achieved, tissue may be easily accessed, organs may be tilted at an ideal angle, or perhaps even to provide comfort and safety for both the patient and the operator.

An elevation mechanism (13) may provide elevation of a mammal and even an angle (14) of elevation. An elevation mechanism may be variable-height platforms, ramps, a tilt table, a sling, steps, boxes, or the like. These may be powered by the patient's own motion, gravity, electric or hydraulic motors, manpower, or the like or may even be unpowered. A positioning device may incorporate padding, a squeezing mechanism, metal, wood, plastic, rubber foam, fabric components or other fabrication materials, or the like, and may be constructed as a single unit or in modular fashion to allow adjustment or even deconstruction.

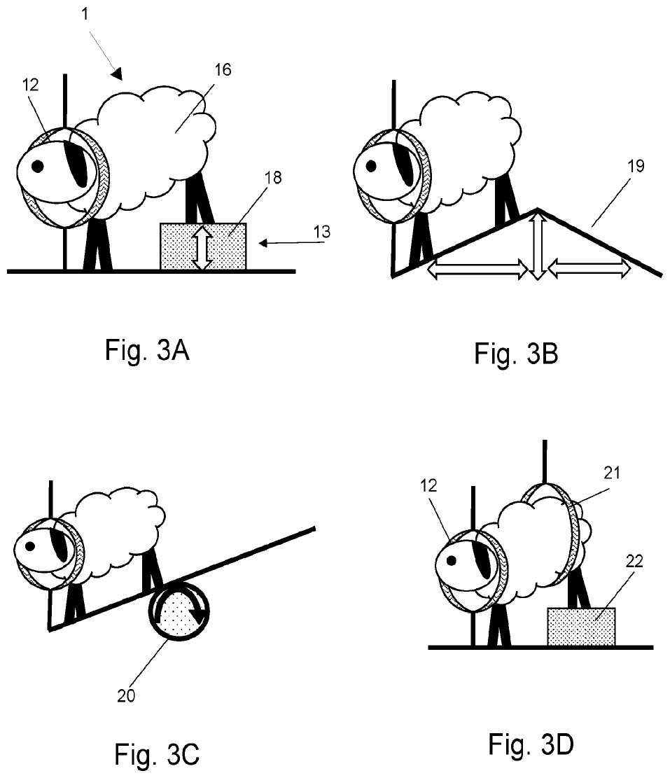

FIGS. 3A-3D shows four non-limiting examples of elevating a mammal by changing a physical angle of the animal. The hips (16) of an ewe can be elevated to modify the angle of the reproductive tract. A mammal positioner (12) may be a restraint device such as a headcatch perhaps such as those found in standard livestock chutes. An elevation mechanism (13) may be a variable-height platform (18), ramp system of variable grade (19), or a tilt-table-like device (20), abdominal sling (21) with foot support (22) as shown.

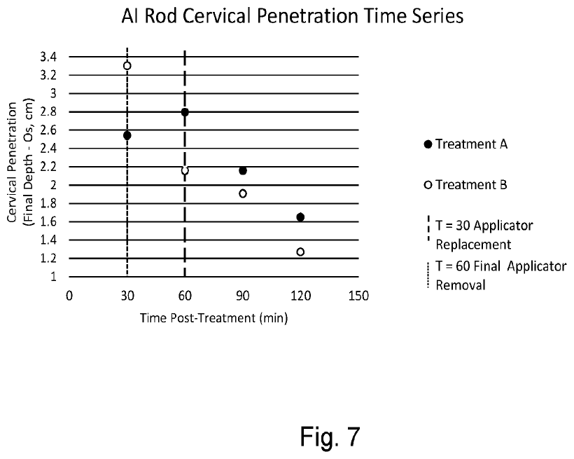

FIG. 8 shows an example of an angle (14) measure of a hip elevation angle (O) formed by the horizontal ground and the body line between the shoulder (17) joint of the mammal and the hip (16) joint. An elevation mechanism may be manual or may be mechanical.

In a non-limiting example, an ewe may be positioned in a squeeze chute and its hips may be elevated: at any angle, between about 5 to about 30 degrees angle, between about 5 and about 20 degrees, between about 5 and about 50 degrees, between about 15 and about 30 degrees, or perhaps even horizontal such that the cervix and uterus may be optimally available for contact and manipulation with no, or minimal damage to the cervix and vaginal area, or the like. In another non-limiting example, this may enable safe passage of an insemination rod or similar device as well as may provide proper positioning of the device within the mammal.

A delineation of an angle of elevation may be independent of a size of a mammal. Measurement of an angle may be provided so that it can be repeatable with the mammal and with other mammals. The elevation of a mammal may provide effective positioning of target tissue for exposure to a treatment, procedure, or the like.

In embodiments, a mammal positioner and even an elevation mechanism may incorporate mechanisms perhaps to allow an operator access to a target tissue from any direction such as below, beside or even behind an animal. A mammal positioner may incorporate illustrations, demarcations, or other illustrations, or the like perhaps to guide the operator in choosing the appropriate settings for the desired angle based on the dimensions of the patient.

A delivery mechanism may include a method for timing the contact of the solution with the appropriate tissue. Timing may include appropriate timing for the tissues, for the specific species, and for the specific application, appropriate timing to achieve the appropriate response, the appropriate timing of treatment as well as the appropriate physiological timing, or the like. A patient can be restrained in such a way as to most easily affect treatment of the target tissue or area perhaps without harm, injury or even pain.

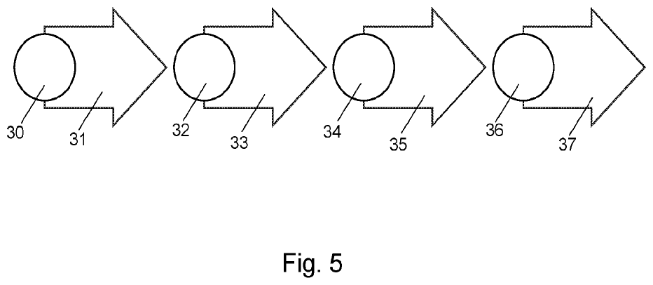

FIG. 5 shows a non-limiting example of a work flow for reducing injury in treated tissues. A treatment may be prepared (30), tissue requirements (31) may be determined, a patient (32) such as a mammal may be prepared, target tissue may be positioned (33), a treatment may be administered (34), the timing of the treatment or treatment applicator may be determined (35), a procedure (36) is executed such as passing into or through an orifice (37).

Embodiments of the application may be used to avoid the requirement for anesthesia, to avoid the use of pain-suppressing medicine, or perhaps even without any chemical intervention. The disclosed embodiments can be used together or independently in a complete system.

All aspects of positioning, delivering, applying, and the like of a treatment may be adjusted whether in length, width, or force depending on the specific application. Such option of adjustment is innately included in this disclosure for each aspect, whether specified or not.

### EXPERIMENT 1: IN VITRO EFFICACY OF SOLUTION

Ewe and bison cervical tracts were obtained to assess local responses in living tissue thereby modeling in vivo response to a solution. Tracts were treated for 1 hour with the respective solution using an absorbent matrix applicator, then ¼ cc AI rods were passed through the cervix, mimicking trans-cervical artificial insemination. A total of 223 tracts were utilized with each of 13 treatments tested in 11 replicates. The estimated number of rings passed was recorded. Post-treatment tracts were dissected, then length and cervical anatomy were recorded, and relationships between cervical structure, ring number and position were and analyzed.

TABLE 1
Definition of extracts utilized in all experimental examples.

| Extract | Primary | Secondary |
|---|---|---|
| Name | Components | Components |
| Control | No solution utilized (but delivery | |
| | device was utilized) | |
| Extract A | glycosylated phenolic compounds, | quercitin, isohamnetin (I) |
| | anthocyanin, carotenoinds, flavonoids | glycoside |
| Extract B | Apocarotenoids, carotenoids, | quercitin, isohamnetin (I) |
| | glycosylated phenolic compounds, | glycoside, triglycerides, |
| | anthocyanin, carotenoids, flavonoids | tocopherols |
| Extract C | apocarotenoids, carotenoids | triglycerides, tocopherols |
| Extract D | apocarotenoids, carotenoids, hyoscine | triglycerides, tocopherols |
| | butylbromide | |
| Extract E | apocarotenoids, glycosylated | quercitin, isohamnetin (I) |
| | phenolic compounds, anthocyanin, | glycoside, triglycerides, |
| | carotenoids, flavonoids, hyoscine | tocopherols |
| | butylbromide | |
| Extract F | hyoscine butylbromide | |
| Extract G | glycosylated phenolic compounds, | quercitin, isohamnetin (I) |
| | anthocyanin, carotenoids, flavonoids, | glycoside |
| | hyoscine butylbromide | |
| Extract H | cholecalciferol | medium chain triglycerides |
| Extract I | linoleic acid, linolenic acid, terpene | oleic acid, palmitic acid, |
| | alcohols, delta-9-tetrahydrocanabinol, | stearic acid, β-sitosterol, |
| | cannabidiol, terpenoids | methyl salicylate, |
| | | tocopherols, carophyllenes |
| Extract J | linoleic acid, linolenic acid, terpene | phenolic acids, flavonoids, |
| | alcohols, oleic acid, terpenoids, | sterols, trace minerals |
| | salicylic acid, gallic acid, ferulic acid, | |
| | vanillic acid, magnesium | |
| Extract K | ricinoleic acid, linoleic acid, oleic | stearic acid, linolenic acid |
| | acid | |
| Extract L | Terpene alcohols, linalyl acetate, | Limonene, camphor, |
| | carophyllene, eucalyptol, lavandulyl | lavandulol, terpenoids |
| | acetate | |
| Extract M | Monoterpenoid phenols, p-cymene, | Limonene, camphene, |
| | γ-terpinene | pinene, carophyllene |
| Extract N | Protease enzymes | |

Ring passage data was transformed into a percentage of rings passed to normalize the data and to allow comparison across an undefined variety of tissues, breeds, ages, and other etiological differences. A paired T-test indicated trends in increased passage of AI rod (P=0.064) when treating the cervix versus control.

TABLE 2
Efficacy of 13 treatments compared to control in vitro using
¼ cc rod on ewe cervices where * indicates
statistically significant improvement in passage over control.

| | Avg. % Cervical |
|---|---|
| Treatment | rings passed |
| Control | 40.24 |
| A | 81.35* |
| B | 53.96* |
| C | 57.15* |
| D | 48.41* |
| E | 50.24* |
| F | 50.02* |
| G | 44.10 |
| H | 42.14 |

### EXPERIMENT 2: COMPATIBILITY WITH SPERM IN VITRO

A successful cervical softening agent should be safe for a mammal's tissues and also for sperm used to inseminate the mammal. It was anticipated that the cervical tract and the AI rod will each retain a residue of the solution treatment which may then combine with the semen inside the cervix or uterus. Therefore, experiments were performed using sperm samples from boars to evaluate the toxicity of each proposed treatment and blends.

Extended boar sperm doses (28×10⁶/ml; 1 ml) were incubated with each of the miscible solutions at 3 concentrations, 0.1 μL, 0.5 μL, or 1 μL per 1 mL extended sperm, then the sperm with the solution were incubated for 3 hours at 37° C. Motility characteristics were measured via computer assisted sperm analyzer. This experiment was replicated 4 times.

TABLE 3
Sperm motility after exposure to three different concentrations
of treatment compounds. Data are expressed as motile percentage
of the total number of spermatozoa after exposure for 3 hours.
There are no statistical differences between control and treatment.

| Dose | | | | | | | | | |
|---|---|---|---|---|---|---|---|---|---|
| (μL in | | | | | | | | | |
| 1 mL | | | | | | | | | |
| sperm) | Ctrl | F | I | K | N | J | C | L | H |
| 0.1 | 89.38 | 90.7 | 89.0 | 89.3 | 87.9 | 91.65 | 90.3 | 88.5 | 86.3 |
| 0.5 | 89.38 | 93.3 | 92.2 | 90.46 | 85.5 | 91.6 | 88.8 | 90.0 | 91.1 |
| 1 | 89.38 | 91.8 | 88.0 | 92.3 | 83.6 | 90.4 | 90.2 | 85.8 | 90.5 |

These data can demonstrate the compatibility of solutions with different types of cells which may underline the safety of the embodiments of the technology and broad applications.

### EXPERIMENT 3: IN VIVO EFFICACY OF APPLICATOR, SOLUTION, AND INSTRUMENT FOR DELIVERY

The cervix of each of 15 ewes were randomly exposed to either one of two treatment solutions, or no solution (control) using a cotton applicator and delivery instrument. Each ewe received an applicator. After 60 minutes, treatment applicator was removed and the ewes were led to a chute then positioned with their hips at an elevation to assist the technician in navigating the vaginal and cervical canals for artificial insemination with a 0.25 cc artificial insemination rod. Upon applicator removal most were found to contain a layer of white mucus and discoloration on the end perhaps indicating contact with a cervical surface. Such discharge can be a common sign of a healthy estrus cycle. The AI rod was passed as deeply into cervix as possible without undue pressure. The depth of the external cervical os and final depth of penetration, was marked on the rod sheath, and the estimated the number of cervical rings passed, was recorded. The external cevical os, the protective entry of the posterior end of the cervix, allows passage between the uterine cavity and vaginal cavity.

Treatment A resulted in a 100% rate of rod passage through the cervix and penetration into the uterus. Using ¼ cc rods, Treatment B had 75.15% passage, while in the control treatment, a rod was able to be passed only 30% of the time. The control rate may be similar to reported success in industry and literature.

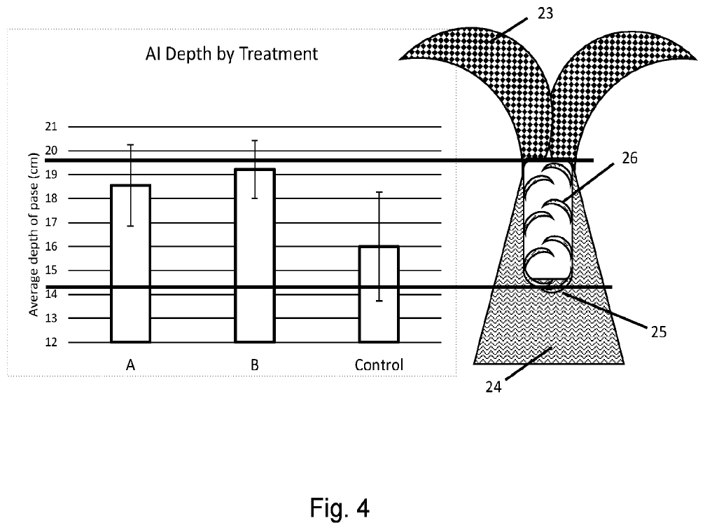

The average depth of penetration for treatments A and B were greater than that for the control (Table 3 and FIG. 4). The average length of an ewe cervix can be about 5.7 cm, though the length of the vaginal cavity can vary between about 11.4 to 17.8 cm, with an average of about 14 cm. Thus, a consistent increase in depth of passage by about 2.5 cm can represent about 20-25% increase in cervical penetration.

In terms of absolute depth of passage of AI rods, Treatment A (mean=20.22 cm, 95% CI [17.27, 20.22]) enabled a statistically significant (p<0.01) increase in depth compared to the control (mean=15.16 cm, 95% CI [12.90, 17.42]). Treatment B (mean=17.7 cm, 95% CI [16.23, 19.17]) is not statistically different from either the control or Treatment A but Treatment B still had a 2.54 cm improvement in rod passage depth over the control.

Like Experiment 1, in 100% of the treated ewes, the external cervical os was passed indicating efficacy of treatment, even when using an ½ cc rod. Moreover, the data may reiterate the ability to deposit semen in the uterine body without invasive laparoscopy.

TABLE 4
Absolute depth of treatment

| | % of cervical | | Absolute depth | |
|---|---|---|---|---|
| Treatment | rings passed | Std dev | passed (cm) | Std dev |
| A | 100 | 0 | 18.5 | 1.02 |
| B | 75.2 | 49.7 | 19.3 | 1.22 |
| Control | 30 | 11.6 | 16.0 | 2.29 |

TABLE 5
AI depth by Treatment. AI depth can be defined as the distance
between final depth and the location of the external os.
Both data aredetermined by the AI technician.

| Treatment | AI rod depth past os (cm) | Std dev |
|---|---|---|
| A | 4.95 | 0.86 |
| B | 6.50 | 0.71 |
| Control | 4.57 | 0.86 |

FIG. 4 provides data from Experiment 3 using one possible embodiment. The penetration depth of an artificial insemination rod into the reproductive tract using ewes in estrus is illustrated as both a bar graph and beside an illustration of a ewe reproductive tract. Vaginal tissue (24) connects to a ˜5.08 cm cervix, external cervical os (26) containing cervical rings and internal cervical os (25) leading into the uterine horns (23).

### EXPERIMENT 4: ½ CC ROD PASSING

Traditionally artificial insemination rods are of two sizes, either 0.25 cc or 0.5 cc. To assess a full impact of dilation, a 0.5 cc artificial insemination rod that can hold either a 0.25 or 0.5 cc straw was used to assess the dilation of the cervix. Ten ewes, 2 control, and 4 for each solution A and B were utilized.

Like Experiment 1, the external cervical os was passed in 100% of the treated ewes which can indicate efficacy of treatment even in non-estrus ewes, and even when using an 0.5 cc rod (Table 5). These data indicate a larger item may be passed, as in other potential uses.

TABLE 6
Average penetration of a 0.5 cc artificial insemination rod

| | | Avg. Cervical | |
|---|---|---|---|
| | Avg. Depth | Distance Passed | Average of |
| Treatment | Passed (cm) | (cm) | % passage |
| Control | 15.16 ± 1.83 | 1.91 ± 2.69 | 40.00 |
| A | 18.75 ± 1.68 | 3.05 ± 0.89 | 88.57 |
| B | 17.70 ± 1.96 | 3.45 ± 2.54 | 88.57 |

### EXPERIMENT 5: IMPACT INDEPENDENT OF PHYSIOLOGICAL STATUS IN PREPARING TISSUES FOR PASSAGE

Sheep are often short day breeders and can be fertile in the autumn and winter with a drastic fertility decline in spring and summer. Tests were conducted to assess the impact of different solutions on ewes that are anestrus, a period when animals are not cycling and do not produce the hormones that may allow dilation of the cervix. In support of non-reproductive applications, anestrous unsynchronized ewes were treated to see if it was possible to create a relaxation event without physiological and hormonal support.

Ewes were used to assess the ability to pass an artificial insemination rod using some embodiments of the application. The ewes were confirmed in anestrus by a lack of response to synchronization hormones.

Nine ewes were treated with solution A. A control (untreated) ewe was not tested here to avoid undue damage to the reproductive tract by trying to pass an artificial insemination rod when there is no physiological or artificial assistance. When using larger rods and lack of estrus, it may be imperative to optimize ewe position. Therefore, the data in the section are further confounded by 3 different techniques to position ewes.

As in Experiment 1, in 100% of the treated ewes, the external cervical os was passed perhaps indicating efficacy of treatment even in non-estrus ewes and when using an ½ cc rod. Data can demonstrate efficacy in situations where dilation is not a biologically mediated event, or perhaps even when dilation is required but unable to be accomplished by physiologically natural means.

TABLE 7
Distance of passing larger artificial insemination rods
in anestrus ewes. Note average depths are reported ±
one standard deviation. Percent of passage is calculated
based on average rings (5) from tracts dissected.

| Average of Depth passed | 13.97 cm |
|---|---|
| StdDev of Depth passed | 1.76 cm |
| Average number of Rings Passed | 1.29 |
| StdDev of number of Rings | 0.96 |
| Passed | |
| Average percentage rings passed | 25.88% |

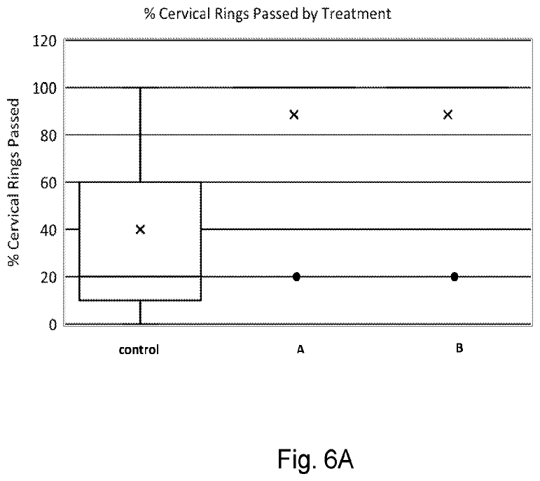

FIG. 6A provides box and whisker plots showing the percentage of total cervical rings passed without (control) or with treatment A or B. In the treated tissues (A and B), the uterine body was penetrated by a ¼ inch AI rod 100% of the time (thickened line at 100). FIG. 6B provides box and whisker plots showing the depth to which a 0.5 cc AI rod was passed within the cervix in Experiment 4. The difference between mean of treated versus untreated (control) is more than 3.81 cm which is 50-75% of the length of a cervical tract. X indicates the median value.

### EXPERIMENT 6: TRANSIENT TREATMENT EFFECT

Ewes in estrus were used to test the period of efficacy. Two ewes were exposed to either treatment A or B (no untreated animals) for 1 hour total. At 30 min, the treatment applicator was removed and an AI rod was passed. A fresh applicator containing the same treatment was then delivered, and rod passed again 30 min later, a total 60 min from the start of the experiment.

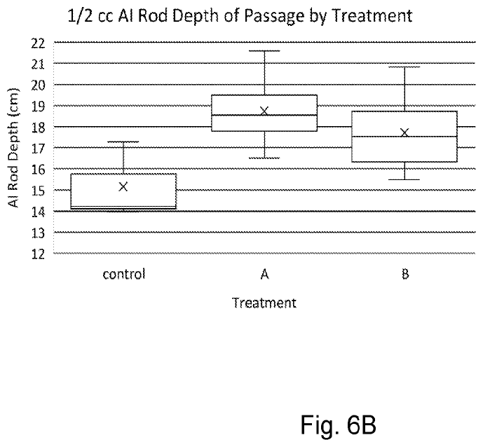

A decline in efficacy was observed over time, as can be understood from FIG. 7. Optimal delivery time for the treatments utilized may appear to occur between about 30 and about 60 minutes. This is a positive response as the treatment can be deemed to be transient.

FIG. 7 shows a time series of attempted passage of an 0.5 cc artificial insemination rod into cervix at 30 minutes intervals during a total elapsed time of 2 hours. Applicators were delivered at T=0, removed at T=30 minutes to allow a rod to be passed, and a fresh applicator delivered which was removed at T=60 minutes and not replaced.

### EXPERIMENT 7: EFFICACY OF SYSTEM AND METHOD TO ATTAIN PREGNANCY

Ewes in estrus were used to test the ability to attain pregnancy. One animal received no device, applicator or solution (the control). Two ewes received cervical treatment with solution A and 2 others with solution B. After 60 minutes of exposure to the treatments, all were inseminated with frozen/thawed ram semen packaged at 200 million sperm/ml. Sperm had a 3 hr post-thaw quality of 48.60% motility and 89.37% membrane intact and acrosome intact. At 45 days post insemination, two treated ewes were pregnant giving a conception rate of 40%. These results provide data that the treatment can be efficacious in achieving pregnancy.

### EXPERIMENT 8: HIP ANGLE/ELEVATION

Two anestrus, unsynchronized ewes were used to determine the effects of a range of hip elevation angles on the depth to which an AI rod could be passed in concert with the tissue treatment. Both ewes received cervical treatment with solution A, and the hips elevated at 0 degrees (all feet flat on the ground) and at two angles between 0 and 30 degrees as may be understood from FIGS. 3A-D and FIG. 8. A moderate angle achieved by elevating hind feet as in FIG. 3A (about 18.4 degrees) resulted an improvement in AI rod passage compared to no elevation (0 degrees).

Experiments 3-8 are all executed in vivo and can show efficacy of the embodiments discussed herein.

While the invention has been described in connection with some preferred embodiments, it is not intended to limit the scope of the invention to the particular form set forth, but on the contrary, it is intended to cover such alternatives, modifications, and equivalents as may be included within the spirit and scope of the invention as defined by the statements of inventions. Examples of alternative claims may include:
1. A tissue treatment system comprising:
   a treatment applicator containing a treatment having a natural substance;
   wherein said treatment is configured to temporarily relax a target tissue of a mammal.
2. The system as described in clause 1 or any other clause wherein said target tissue is chosen from is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
3. The system as described in clause 1 or any other clause wherein said mammal is chosen from: an ungulate, small ruminant animal, ewe, doe, elk, and cow.
4. The system as described in clause 1 or any other clause wherein said natural substance comprises a plant-based or algae-based substance.
5. The system as described in clause 4 or any other clause wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.
6. The system as described in clause 1 or any other clause wherein said treatment further comprises a substance chosen from: glycosylated phenolic compounds, anthocyanin, carotenoids, flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.
7. The system as described in clause 1 or any other clause and further comprising a procedure conducted on said mammal chosen from: artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.
8. The system as described in clause 1 or any other clause wherein said treatment is configured to temporarily expand and allow passage through a restricted orifice of said mammal.
9. The system as described in clause 1 or any other clause wherein said treatment is in a physical state chosen from: liquid, semi-solid, solid, gel, cream, capsule, suppository, poultice, bioadhesive tablet, patch, film, ring, foam, polymer, mucoadhesive gel, and any combination or permutation thereof.
10. The system as described in clause 1 or any other clause wherein said treatment applicator is chosen from: cotton, polyester, a natural material, a synthetic material, absorbent swab, cushion, sponge, sea sponge, pad, diaphragm, rubber convex device, pessary, seaweed, and any combination or permutation thereof.
11. The system as described in clause 1 or any other clause wherein said treatment is in a capsule made of a material chosen from: wax, sugar, gel, glycerin, cellulose, collagen, and any combination or permutation thereof.
12. The system as described in clause 1 or any other clause wherein said treatment applicator is configured to topically apply said treatment to said target tissue for an amount of time chosen from: about 15 minutes, about 30 minutes, about 60 minutes, and about 180 minutes.
13. The system as described in clause 1 or any other clause and further comprising a delivery mechanism configured to apply said treatment or a treatment applicator containing said treatment to said target tissue, said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.
14. The system as described in clause 13 or any other clause wherein said delivery mechanism is configured to apply said treatment or said treatment applicator containing said treatment on or near said target tissue.
15. A method for preparing tissues comprising the steps of:
   providing a target tissue of a mammal;
   topically applying a treatment comprising a natural substance to said target tissue; and temporarily relaxing said target tissue with said treatment.
16. The method as described in clause 15 or any other clause wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
17. The method as described in clause 15 or any other clause wherein said mammal is chosen from: an ungulate, small ruminant animal, ewe, doe, and cow.
18. The method as described in clause 15 or any other clause wherein a physiological response to said treatment of said mammal is not dependent on hormonal influences.
19. The method as described in clause 15 or any other clause wherein said natural substance comprises a plant-based or algae-based substance.
20. The method as described in clause 19 or any other clause wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.
21. The method as described in clause 15 or any other clause wherein said treatment further comprises a substance chosen from: glycosylated phenolic compounds, anthocyanin, carotenoids, flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.
22. The method as described in clause 15 or any other clause wherein said step of temporarily relaxing said target tissue with said treatment comprises a step of relaxing said target tissue for a period of time chosen from up to about 20 minutes, up to about 30 minutes, up to about 60 minutes, and up to about 180 minutes.
23. The method as described in clause 1 or any other clause and further comprising a step of treating said mammal having said temporarily relaxed target tissue with a procedure chosen from: artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.
24. The method as described in clause 15 or any other clause wherein said step of temporarily relaxing said target tissue with said treatment comprises a step of temporarily expanding and allowing passage through a restricted orifice of said mammal.
25. The method as described in clause 15 or any other clause wherein said treatment is in a physical state chosen from: liquid, semi-solid, solid, gel, cream, capsule, suppository, poultice, bioadhesive tablet, patch, film, ring, foam, polymer, mucoadhesive gel, and any combination or permutation thereof.
26. The method as described in clause 15 or any other clause wherein said step of topically applying said treatment comprising said natural substance to said target tissue comprises a step of topically applying said treatment comprising said natural substance to said target tissue with a treatment applicator.
27. The method as described in clause 26 or any other clause wherein said treatment applicator is chosen from: cotton, polyester, a natural material, a synthetic material, absorbent swab, cushion, sponge, sea sponge, pad, diaphragm, rubber convex device, pessary, capsule, and any combination or permutation thereof.
28. The method as described in clause 15 or 27 or any other clause and further comprising a step of providing said treatment in a capsule made of a material chosen from: wax, sugar, gel, glycerin, cellulose, collagen, and any combination or permutation thereof.
29. The method as described in clause 15 or any other clause wherein said step of topically applying said treatment comprising said natural substance to said target tissue comprises a step of topically applying said treatment to said target tissue for an amount of time chosen from: about 15 minutes, about 30 minutes, about 60 minutes, and about 180 minutes.
30. The method as described in clause 15 or any other clause wherein said step of topically applying said treatment comprising said natural substance to said target tissue comprises a step wherein said step of topically applying said treatment to said target tissue with a delivery mechanism, said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.
31. The method as described in clause 30 or any other clause and further comprising a step of applying said treatment from said delivery mechanism on or near said target tissue.
32. The method as described in clause 15 or any other clause and further comprising a step of positioning said mammal before said step of topically applying said treatment to said target tissue.
33. The method as described in clause 32 or any other clause wherein said step of positioning said mammal comprises a step chosen from:
   elevating hips of said mammal;
   elevating hips of said mammal above its shoulders between about 5 to about 30 degrees;
   elevating hips of said mammal above its shoulders between about 5 to about 20 degrees;
   elevating hips of said mammal above its shoulders between about 5 to about 50 degrees; and
   elevating hips of said mammal above its shoulders between about 15 to about 30 degrees.
34. A treatment configured to temporarily relax a target tissue of a mammal, said treatment comprises a natural substance.
35. The treatment as described in clause 34 or any other clause wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
36. The treatment as described in clause 34 or any other clause wherein said mammal is chosen from: an ungulate, small ruminant animal, ewe, doe, and cow.
37. The treatment as described in clause 34 or any other clause wherein a physiological response to said treatment of said mammal is not dependent on hormonal influences.
38. The treatment as described in clause 34 or any other clause wherein said natural substance comprises a plant-based or algae-based substance.
39. The treatment as described in clause 38 or any other clause wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.
40. The treatment as described in clause 34 or any other clause wherein said treatment further comprises a substance chosen from: glycosylated phenolic compounds, anthocyanin, carotenoids, flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.
41. The treatment as described in clause 34 or any other clause wherein said treatment is configured to temporarily relax said target tissue for a period of time chosen from up to about 20 minutes, up to about 30 minutes, up to about 60 minutes, and up to about 180 minutes.
42. The treatment as described in clause 34 or any other clause and further comprising a procedure conducted on said mammal chosen from: artificial insemination, retrieval of oocytes, retrieval of embryos, transfer of oocytes, transfer of embryos, delivery of sperm cells, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.
43. The treatment as described in clause 34 or any other clause wherein said treatment is configured to temporarily expand and allow passage through a restricted orifice of said mammal.
44. The treatment as described in clause 15 or any other clause wherein said treatment is in a physical state chosen from: liquid, semi-solid, solid, gel, cream, capsule, suppository, poultice, bioadhesive tablet, patch, film, ring, foam, polymer, mucoadhesive gel, and any combination or permutation thereof.
45. The treatment as described in clause 34 or any other clause and further comprising a treatment applicator configured to topically apply said treatment to said target tissue.
46. The treatment as described in clause 45 or any other clause wherein said treatment applicator is chosen from: cotton, polyester, a natural material, a synthetic material, absorbent swab, cushion, sponge, sea sponge, pad, diaphragm, rubber convex device, pessary, seaweed, and any combination or permutation thereof.
47. The treatment as described in clause 34 or any other clause wherein said treatment is in a capsule made of a material chosen from: wax, sugar, gel, glycerin, cellulose, collagen, and any combination or permutation thereof.
48. The treatment as described in clause 45 or any other clause wherein said treatment applicator is configured to topically apply said treatment to said target tissue for an amount of time chosen from: about 15 minutes, about 30 minutes, about 60 minutes, and about 180 minutes.
49. The treatment as described in clause 34 or any other clause and further comprising a delivery mechanism configured to apply said treatment or a treatment applicator containing said treatment to said target tissue, said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.
50. The treatment as described in clause 49 or any other clause wherein said delivery mechanism is configured to apply said treatment or said treatment applicator containing said treatment on or near said target tissue.
51. The treatment as described in clause 34 or any other clause and further comprising a mammal positioner.
52. The treatment as described in clause 51 or any other clause wherein said mammal positioner is configured to position said mammal chosen from:
   elevating hips of said mammal;
   elevating hips of said mammal above its shoulders between about 5 to about 30 degrees;
   elevating hips of said mammal above its shoulders between about 5 to about 20 degrees
   elevating hips of said mammal above its shoulders between about 5 to about 50 degrees; and
   elevating hips of said mammal above its shoulders between about 15 to about 30 degrees.
53. A method for treating mammals comprising the steps of:
   providing a mammal;
   providing a target tissue of said mammal;
   elevating hips of said mammal above its shoulders to position said target tissue of said mammal, wherein said hips are elevated at an angle between about 5 to about 50 degrees above said shoulders; and
   applying a treatment to said target tissue.
54. The method as described in clause 53 or any other clause wherein said mammal comprises a four-legged mammal.
55. The method as described in clause 53 or any other clause wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
56. The method as described in clause 53 or any other clause wherein said hips are elevated at an angle between about 15 to about 30 degrees above said shoulders.
57. The method as described in clause 53 or any other clause wherein said mammal is chosen from: an ungulate, small ruminant animal, ewe, doe, and cow.
58. The method as described in clause 53 or any other clause wherein said step of elevating said hips of said mammal above its shoulders to position said target tissue of said mammal comprises a step of elevating said hips of said mammal above its shoulders to position said target tissue of said mammal with an elevation mechanism chosen from:
   variable-height platforms, ramps, a tilt table, slings, steps, boxes and any combination or permutation thereof.
59. The method as described in clause 58 or any other clause wherein said step of elevating said hips of said mammal above its shoulders to position said target tissue of said mammal comprises a step of manually elevating said hips of said mammal above its shoulders to position said target or mechanically elevation mechanism elevating said hips of said mammal above its shoulders to position said target tissue.
60. The method as described in clause 58 or any other clause and further comprising a step of delineating said angle of elevation independent of a size of said mammal.
61 The method as described in clause 53 or any other clause and further comprising a step of measuring said angle so that it is repeatable with said mammal and with other mammals.
62. The method as described in clause 53 or any other clause wherein said step of elevating said hips of said mammal above its shoulders to position said target tissue of said mammal comprises a step of effectively positioning said target tissue of said mammal for exposure to said treatment.
63. A mammal treatment system comprising:
   an elevation mechanism configured to elevate hips of a mammal above its shoulders to expose a target tissue for treatment of mammal;
   wherein said elevation mechanism is configured to elevate said hips at an angle between about 5 to about 50 degrees above said shoulders.
64. The system as described in clause 63 or any other clause wherein said mammal comprises a four-legged mammal.
65. The system as described in clause 63 or any other clause wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
66. The system as described in clause 63 or any other clause wherein elevation mechanism is configured to elevate said hips at an angle between about 15 to about 30 degrees above said shoulders.
67. The system as described in clause 63 or any other clause wherein said mammal is chosen from: an ungulate, small ruminant animal, ewe, doe, elk, and cow.
68. The system as described in clause 63 or any other clause wherein said elevation mechanism is chosen from: variable-height platforms, ramps, a tilt table, a sling, steps, boxes and any combination or permutation thereof.
69 The system as described in clause 68 or any other clause wherein said elevation mechanism comprises a manual elevation mechanism or a mechanical elevation mechanism.
70. A method for treating tissues comprising the steps of:
   providing a target tissue of a mammal;
   providing a treatment applicator containing a treatment having a natural substance;
   topically applying said treatment having said natural substance to said target tissue from contact of said applicator with said target tissue;
   removing said applicator from said target tissue of said mammal; and
   temporarily relaxing said target tissue with said treatment.
71. The method as described in clause 70 or any other clause wherein said target tissue is chosen from is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
72. The method as described in clause 70 or any other clause wherein said mammal is chosen from: an ungulate, small ruminant animal, ewe, doe, elk, and cow.
73. The method as described in clause 70 or any other clause wherein a physiological response to said treatment of said mammal is not dependent on hormonal influences.
74. The method as described in clause 70 or any other clause wherein said natural substance comprises a plant-based or algae-based substance.
75. The method as described in clause 74 or any other clause wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.
76. The method as described in clause 70 or any other clause wherein said treatment further comprises a substance chosen from: glycosylated phenolic compounds, anthocyanin, carotenoids, flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.
77. The method as described in clause 70 or any other clause wherein said step of temporarily relaxing said target tissue with said treatment comprises a step of relaxing said target tissue for a period of time chosen from up to about 20 minutes, up to about 30 minutes, up to about 60 minutes, and up to about 180 minutes.
78. The method as described in clause 70 or any other clause and further comprising a step of treating said mammal having said temporarily relaxed target tissue with a procedure chosen from: artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.
79. The method as described in clause 70 or any other clause wherein said step of temporarily relaxing said target tissue with said treatment comprises a step of temporarily expanding and allowing passage through a restricted orifice of said mammal.
80. The method as described in clause 70 or any other clause wherein said treatment is in a physical state chosen from: liquid, semi-solid, solid, gel, cream, capsule, suppository, poultice, bioadhesive tablet, patch, film, ring, foam, polymer, mucoadhesive gel, and any combination or permutation thereof.
81. The method as described in clause 70 or any other clause wherein said treatment applicator is chosen from: cotton, polyester, a natural material, a synthetic material, absorbent swab, cushion, sponge, sea sponge, pad, diaphragm, rubber convex device, pessary, seaweed, and any combination or permutation thereof.
82 The method as described in clause 70 or any other clause and further comprising a step of providing said treatment in a capsule made of a material chosen from wax, sugar, gel, glycerin, cellulose, collagen, and any combination or permutation thereof.
83. The method as described in clause 70 or any other clause wherein said step of topically applying said treatment comprising said natural substance to said target tissue a step of topically applying said treatment to said target tissue for an amount of time chosen from: about 15 minutes, about 30 minutes, about 60 minutes, and about 180 minutes.
84. The method as described in clause 70 or any other clause wherein said step of topically applying said treatment comprising said natural substance to said target tissue comprises a step wherein said step of topically applying said treatment to said target tissue with a delivery mechanism, said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.
85. The method as described in clause 84 or any other clause further comprising a step of applying said treatment from said delivery mechanism on or near said target tissue.
86 The method as described in clause 70 or any other clause and further comprising a step of positioning said mammal to enable said treatment.
87. A method for relaxing tissues comprising the steps of:
   providing a target tissue of a mammal;
   providing a treatment applicator containing a treatment having a natural substance;
   holding said treatment applicator containing said treatment with a delivery mechanism;
   placing said treatment applicator containing said treatment on or near said target tissue with said delivery mechanism;
   topically applying said treatment to said target tissue from contact of said treatment applicator with said target tissue;
   removing said treatment applicator from said target tissue of said mammal with said delivery mechanism; and
   temporarily relaxing said target tissue with said treatment.
88. The method as described in clause 87 or any other clause and further comprising a step of elevating hips of said mammal above its shoulders to expose said target tissue of said mammal.
89. The method as described in clause 87 or any other clause wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
90. The method as described in clause 87 or any other clause wherein said mammal is chosen from: an ungulate, small ruminant animal, ewe, doe, elk, and cow.
91. The method as described in clause 87 or any other clause wherein said natural substance comprises a plant-based or algae-based substance.
92. The method as described in clause 91 or any other clause wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.
93. The method as described in clause 87 or any other clause wherein said treatment further comprises a substance chosen from: glycosylated phenolic compounds, anthocyanin, carotenoids, flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.
94. The method as described in clause 87 or any other clause and further comprising a step of treating said mammal having said temporarily relaxed target tissue with a procedure chosen from: artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.
95. The method as described in clause 87 or any other clause wherein said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.
96. A tissue treatment system comprising:
   a treatment applicator containing a treatment having a natural substance; and
   a delivery mechanism configured to place said treatment applicator on or near a target tissue of a mammal;
   wherein said treatment is configured to temporarily relax said target tissue.
97. The system as described in clause 96 or any other clause and further comprising an elevation mechanism configured to elevate hips of a mammal above its shoulders to expose a target tissue for treatment of mammal.
98. The system as described in clause 96 or any other clause wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.
99 The system as described in clause 96 or any other clause wherein said mammal is chosen from: an ungulate, small ruminant animal, ewe, doe, elk, and cow.
100. The system as described in clause 96 or any other clause wherein said natural substance comprises a plant-based or algae-based substance.

101. The system as described in clause 100 or any other clause wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.
102. The system as described in clause 96 or any other clause wherein said treatment further comprises a substance chosen from: glycosylated phenolic compounds, anthocyanin, carotenoids. flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.
103. The system as described in clause 96 or any other clause and further comprising a procedure conducted on said mammal chosen from: artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.
104. The system as described in clause 96 or any other clause wherein said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.

As can be easily understood from the foregoing, the basic concepts of the various embodiments of the present invention(s) may be embodied in a variety of ways. It involves both tissue relaxation techniques as well as devices to accomplish the appropriate tissue relaxation. Devices may be used to enhance the functionality of the relaxation. In this application, the tissue relaxation techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the various embodiments of the invention(s) and may not explicitly show how each feature or element can actually be representative of a broader function or of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc. as well as terms that encompass largely but not wholly that which is specified. Further, for this application if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified). In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person of ordinary skill in this field. Where the application is described in device-oriented terminology, each element of the device implicitly performs a function. Apparatus claims may not only be included for the device described, but also method or process claims may be included to address the functions of the embodiments and that each element performs. Neither the description nor the terminology is intended to limit the scope of the claims that will be included in any subsequent patent application.

It should also be understood that a variety of changes may be made without departing from the essence of the various embodiments of the invention(s). Such changes are also implicitly included in the description. They still fall within the scope of the various embodiments of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of embodiments of the invention(s) both independently and as an overall system.

Further, each of the various elements of the embodiments of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the various embodiments of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments of the invention(s) is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “positioner” should be understood to encompass disclosure of the act of “positioning”—whether explicitly discussed or not—and, conversely, were there effectively disclosure of the act of “positioning”, such a disclosure should be understood to encompass disclosure of a “positioner” and even a “means for positioning.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components, programming, subroutines, logic, or elements that are configured to, or configured and arranged to, provide or even achieve a particular result, use, purpose, situation, function, or operation, or as components that are capable of achieving a particular activity, result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed herein and below and in any information statement filed with the application are hereby appended and hereby incorporated by reference, however, as to each of the above, to the extent that such information or statements incorporated by reference might be considered inconsistent with the patenting of the various embodiments of invention(s) such statements are expressly not to be considered as made by the applicant(s).

### U.S. PATENTS

| Patent No. | Kind Code | Date Issued | Patentee |
|---|---|---|---|
| 6758840 | B2 | 2004 Jul. 6 | Knox |

### NON PATENT LITERATURE

United States Provisional Patent Application No. 63/319,866 filed Mar. 15, 2022. First
named inventor: Scharf
Ivanka B.R. Candappa and Pawel M. Bartlewski, A Review of Advances in Artificial
Insemination (AI) and Embryo Transfer (ET) in Sheep, with the Special Reference to
Hormonal Induction of Cervical Dilation and its Implications for Controlled Animal
Reproduction and Surgical Techniques, The Open Reproductive Science Journal, 2011, 3,
162-175
Sukanya Leethongdee, Induction of Cervical Relaxation for Artificial Insemination in
Sheep, Leethongdee S./Thai J Vet Med. 2011. 41(4): 403-407
Luciana C. Padilha-Nakaghi, et al., Local α1-adrenergic blockers: An alternative for sheep
cervix dilation? Animal Reproduction Science 222 (2020) 106609
K. Perry et al. Intracervical application of hyaluronan improves cervical relaxation in the
cwe, Theriogenology 74 (2010) 1685-1690
DEVICES AND METHODS FOR THE TREATMENT OF TISSUE Apr 5, 2021 - Fractyl
Laboratories, Inc., https://patents.justia.com/patent/20220054180#claims. Retrieved
Apr. 5, 2021. 87 pages.

Thus, the applicant(s) should be understood to have support to claim and make claims to embodiments including at least: i) each of the tissue relaxing devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrases “comprising”, “including”, “containing”, “characterized by” and “having” are used to maintain the “open-end” claims herein, according to traditional claim interpretation including that discussed in MPEP § 2111.03. Thus, unless the context requires otherwise, it should be understood that the terms “comprise” or variations such as “comprises” or “comprising”, “include” or variations such as “includes” or “including”, “contain” or variations such as “contains” and “containing”, “characterized by” or variations such as “characterizing by”, “have” or variations such as “has” or “having”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 9 or any other claim” or the like, it could be re-drafted as dependent on claim 1, claim 8, or even claim 11 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A tissue treatment system comprising:
a treatment applicator containing a treatment having a natural substance;
wherein said treatment is configured to temporarily relax a target tissue of a mammal.

2. The system as described in claim 1 wherein said target tissue is chosen from is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.

3. The system as described in claim 1 wherein said mammal is chosen from: an ungulate, small ruminant animal, ewe, doe, elk, and cow.

4. The system as described in claim 1 wherein said natural substance comprises a plant-based or algae-based substance.

5. The system as described in claim 4 wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.

6. The system as described in claim 1 wherein said treatment further comprises a substance chosen from: glycosylated phenolic compounds, anthocyanin, carotenoids, flavonoids, apocarotenoids, quercitin, isohamnetin (l) glycoside, triglycerides, tocopherols, salicylate, linoleic and linolenic acids, phenols, antimicrobial or antifungal agents, anti-inflammatory agents, antioxidants, and any combination or permutation thereof.

7. The system as described in claim 1 and further comprising a procedure conducted on said mammal chosen from: artificial insemination, delivery of sperm cells, retrieval of oocytes, retrieval of embryos, transfer of oocytes, transfer of embryos, intrauterine device placement, surgery, parturition, uterine procedures, uterine examinations, vaginal procedures, cervical semen deposition, laparoscopic artificial insemination, semen deposition, dystocia treatment, dilation procedures, esophageal procedures, occlusion procedures, prostate procedures, passage of renal calculi, passage of a salivary stone, passage of a pyloric sphincter, tissue examinations, insertion of breathing or feeding tubes, inspection of sinuses, and laparoscopic surgery.

8. The system as described in claim 1 wherein said treatment is configured to temporarily expand and allow passage through a restricted orifice of said mammal.

9. The system as described in claim 1 wherein said treatment is in a physical state chosen from: liquid, semi-solid, solid, gel, cream, capsule, suppository, poultice, bioadhesive tablet, patch, film, ring, foam, polymer, mucoadhesive gel, and any combination or permutation thereof.

10. The system as described in claim 1 wherein said treatment applicator is chosen from: cotton, polyester, a natural material, a synthetic material, absorbent swab, cushion, sponge, sea sponge, pad, diaphragm, rubber convex device, pessary, seaweed, and any combination or permutation thereof.

11. The system as described in claim 1 wherein said treatment is in a capsule made of a material chosen from: wax, sugar, gel, glycerin, cellulose, collagen, and any combination or permutation thereof.

12. The system as described in claim 1 wherein said treatment applicator is configured to topically apply said treatment to said target tissue for an amount of time chosen from: about 15 minutes, about 30 minutes, about 60 minutes, and about 180 minutes.

13. The system as described in claim 1 and further comprising a delivery mechanism configured to apply said treatment or a treatment applicator containing said treatment to said target tissue, said delivery mechanism is chosen from: spray bottle, aerosolizer, mist device, syringe, tweezers, forceps, bimanual insertion, tenaculum forceps, pincher device, plunger device, clasping device, speculum, scoop, swab, an adjustable device, and any combination or permutation thereof.

14. The system as described in claim 13 wherein said delivery mechanism is configured to apply said treatment or said treatment applicator containing said treatment on or near said target tissue.

15. A method for preparing tissues comprising the steps of:
providing a target tissue of a mammal;
topically applying a treatment comprising a natural substance to said target tissue; and
temporarily relaxing said target tissue with said treatment.

16. The method as described in claim 15 wherein said target tissue is chosen from: constricted orifice, constricted tissue, muscle tissue, reproductive tissue, ductal tissue, sphincter tissue, mouth, anus, vagina, cervix, uterus, epithelial tissue, organ, esophagus, prostate, renal tissues, urethra, ureter, pyloric sphincter, ducts, tear ducts, scar tissue, venous tissue, and arterial tissue.

17. The method as described in claim 15 wherein said mammal is chosen from: an ungulate, small ruminant animal, ewe, doe, and cow.

18. The method as described in claim 15 wherein a physiological response to said treatment of said mammal is not dependent on hormonal influences.

19. The method as described in claim 15 wherein said natural substance comprises a plant-based or algae-based substance.

20. The method as described in claim 19 wherein said plant-based or algae-based substance comprises extracts derived from families or orders of plants or algae chosen from: Onagraceae, Bixaceae, Rosaceae, Elaeagnaceae, Ericaceae, Rutaceae, Cupressaceae, Lamiaceae, Rosales, Aquifoliaceae, Solancaceae, Lamiales, Rhodophyta, Phaeophyta, Chlorophyta, red seaweed, green seaweed, brown seaweed, and any combination or permutation thereof.

21-104. (canceled)
